(19) United States
(12) Patent Application Publication
(10) Pub. No.: US 2023/0276872 A1
(43) Pub. Date: Sep. 7, 2023

(54) HEAD STRAP SYSTEM FOR EYEWEAR

(71) Applicant: Raymond Fung, Berkeley, CA (US)

(72) Inventor: Raymond Fung, Berkeley, CA (US)

(21) Appl. No.: 18/315,442

(22) Filed: May 10, 2023

(51) Int. Cl. A41D 13/11

(52) U.S. Cl. CPC A41D 13/1184; A41D 13/1161

(57) ABSTRACT

A head strap system is designed to secure eye wear, such as a sleep mask, with optimal fit, comfort, and stability. The system provides over-ear straps and under-ear straps. On each side of the head, the over-ear strap and under-ear strap are each connected to a strap attachment, which is detachably and adjustably secured to a temple pad on the eye wear. The over-ear straps are each connected to an adjustable central elastic band. The under-ear straps are connected to left and right neck straps, which are temporarily secured together in order to wear the head strap system. The over-ear straps may be configured as arcs. An optional chin strap and/or hairline strap further secure the eyewear.

### 1. FIELD OF THE INVENTION

This invention is in the field of straps and attachments, specifically for eyewear.

### 2. BACKGROUND OF THE INVENTION

A sleep mask is used to shield the eyes from light, especially as a sleeping aid that prevents users from awakening prematurely due to the sun or other light sources. The ideal sleep mask is able to remain on the user's face without falling off before this intended wake-up time. Typical causes of fallen sleep masks include head movements during sleep and unconscious removal due to discomfort. This is commonly the result of poor fitting or irritating straps around the ears. A soft mask that fits well without pinching will be superior to a mask that slips off or chafes the face.

### 3. DESCRIPTION OF RELATED TECHNOLOGY

Sleep mask innovations tend to focus on the eyewear. For instance, Schwarz and Zhang describe an eyeshade with modular replaceable eye covers in U.S. Pat. No. 10,646,377.

The head strap is an important and oft-neglected feature of the sleep mask. To the inventor's knowledge, sleep masks in production today use a fixed single head strap, which is to be placed above the ears and is not adjustable beyond the length of the strap. The fixed single-strap structure creates problems with fit, stability, or comfort, and it limits adjustability for various users with different eye and ear shape, placement, and size.

### 4. SUMMARY OF THE INVENTION

The present invention is a head strap system for eyewear, with innovative attachment and adjustment features. It may be used with a sleep mask or other eyewear items such as safety goggles or ski masks. A preferred embodiment has a double-strap structure, with over-ear straps and under-ear straps. In this way, the head strap system is designed to fully surround the ears. Contrast this with prior art systems, which have only one attachment point on each end of the eyewear.

An alternative embodiment features a triple-strap structure, which adds a chin strap to the over-ear and under-ear straps. A further embodiment includes a hairline strap. The double, triple, or quadruple strap system greatly improves adjustability and stability for the sleep mask. The hairline strap and chin strap are secured to the eyewear with removable attachment means such as hook-and-loop fabric. This allows maximal adjustment of the straps. For instance, the point of attachment of the straps to the eyewear is easily adjustable along the vertical or sagittal axes to accommodate different sizes of heads and ears. The angles of attachment are also easily adjustable to accommodate people with ears located lower or higher on the head.

### 5. BRIEF DESCRIPTION OF THE DRAWINGS

FIG. 1A shows the present invention assembled and positioned on a human head

FIG. 1B is an exploded view of FIG. 1, with the components separated for easier visualization and to illustrate the points of attachment/separation.

FIG. 2 is a rear view of the innovative head strap system.

FIG. 3 shows the points of attachment connecting the eyewear, temple pad, and head strap system.

FIG. 4 is a side view of an embodiment of the invention that includes a strap arc and a hairline strap.

FIGS. 5A, 5B, and 5C are three alternative depictions of the head strap system juxtaposed to demonstrate its adjustability.

FIG. 6 is a rear view of the head strap system with snapback straps.

### 6. DETAILED DESCRIPTION OF THE PREFERRED EMBODIMENTS

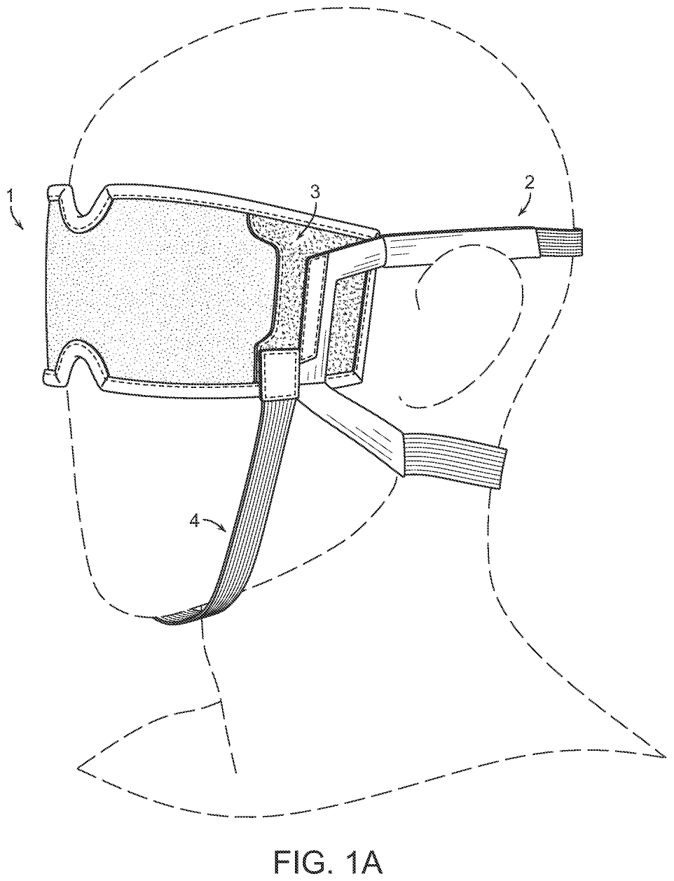

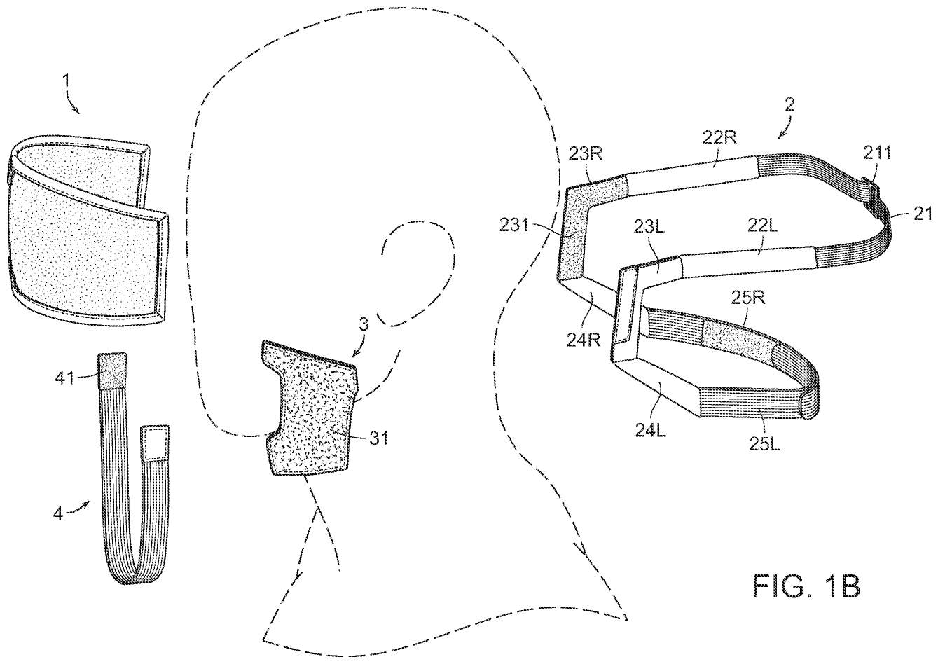

The major components of the sleep mask invention are most clearly seen in FIG. 1B: the eyewear 1, the head strap system 2, the temple pad 3, and the chin strap 4. Detachable securing means, such as hook-and-loop fabrics or snapback straps, enable these four components to be assembled together on the face, as shown in FIG. 1A.

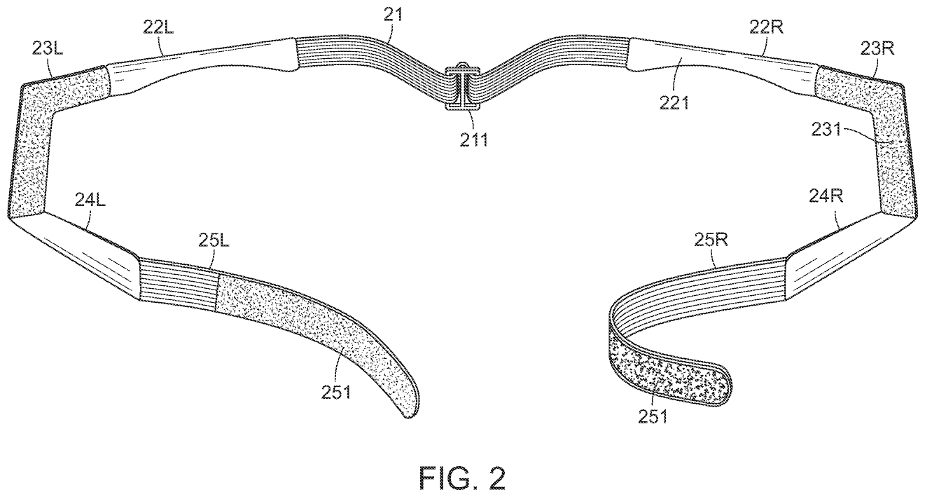

The head strap system 2 is shown in greater detail in FIG. 2. A central elastic band 21 is designed for placement at the back of the head. The central elastic band may include adjustment means 211, such as an adjustment buckle, to make the band longer or shorter.

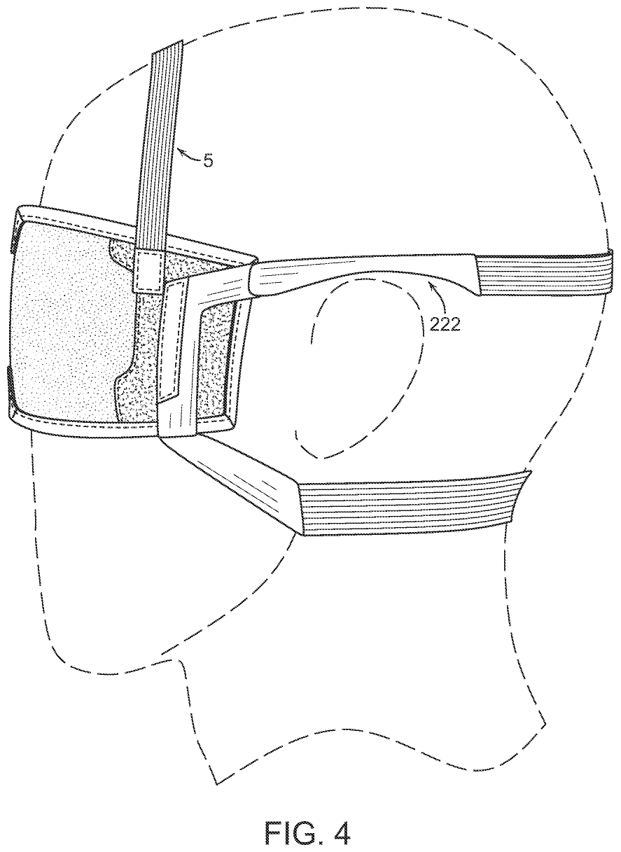

A left over-ear strap 22L and a right over-ear strap 22R are attached end-to-end to the central elastic band. When properly placed, the over-ear straps are designed to protrude forward on the head and over the ears. Each over-ear strap may be configured with a strap arc 222, as shown in FIG. 4. The strap arcs position the over-ear straps completely over the ears, instead of passing between the sides of the head and the pinnae (like the stems of eyeglasses). This presents obvious advantages for comfort, especially when one side of the head is resting on a pillow. The over-ear straps may be formed of any suitable construction material such as plastic, silicone, or fabric, or any combination of such materials. For example, each over-ear strap may have an internal plastic insert covered by a strap cover, 221, optimally a cotton memory foam or other soft material. Alternatively, the over-ear straps may be constructed entirely of silicone or entirely of fabric.

Each over-ear strap is attached to a strap attachment (23L and 23R) at approximately a right angle to the over-ear strap. The strap attachments will be positioned in front of the ears, at the temples. They are designed to attach to the temple pad 3 with detachable securing means 231 such as hook-and-loop fabric.

Continuing along the length of the head strap system 2, around the ears, each strap attachment (23L and 23R) is attached end-to-end to an under-ear strap (24L and 24R). The under-ear straps are ideally made of a heavy elastic material.

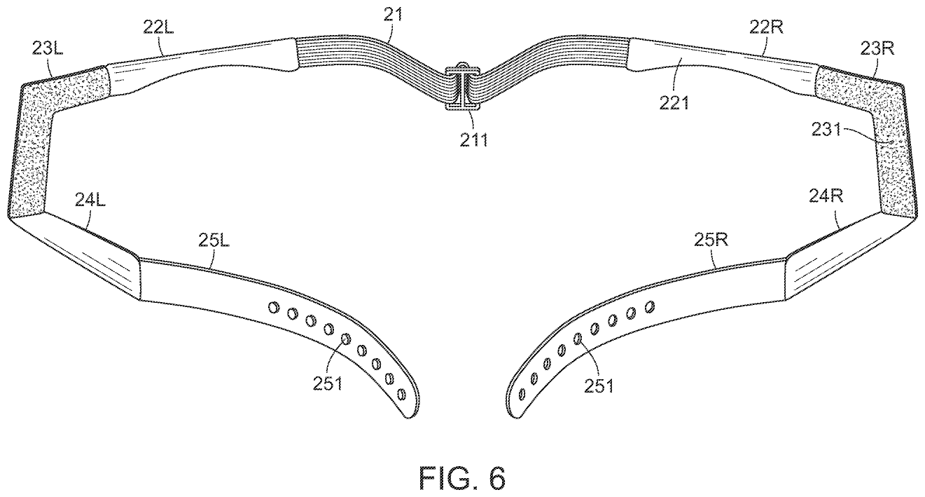

Each under-ear strap (24L and 24R) is joined end-to-end with a neck strap (25L and 25R). The neck straps are designed to lead around the back of the neck. The neck straps are ideally made of neoprene or a similar material. The left neck strap 25L is attachable to the right neck strap 25R with temporary attachment means 251, such as hook-and-loop fabric, a clasp, or snapback straps, as shown in FIG. 6.

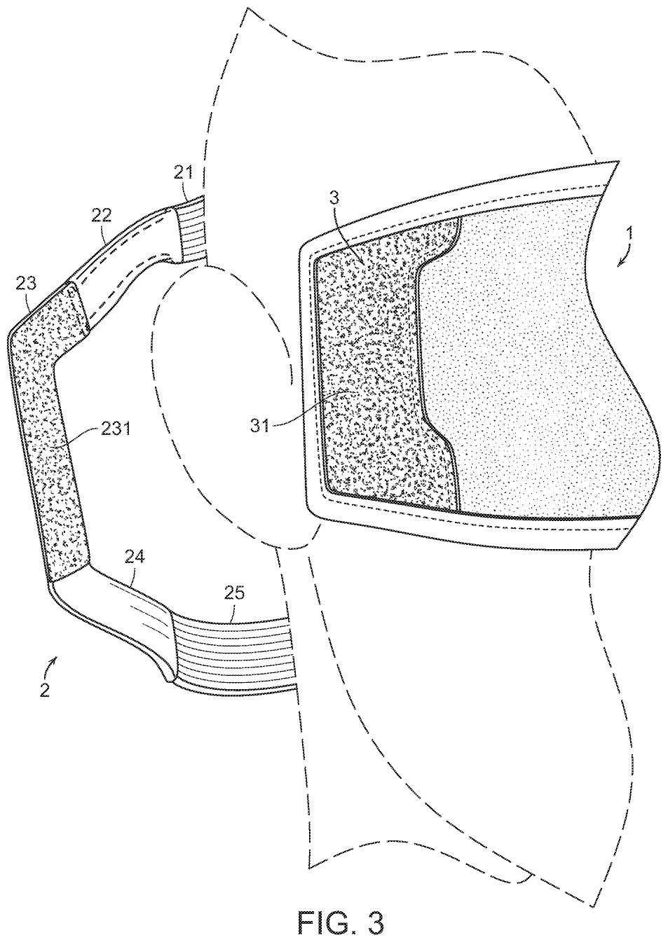

FIG. 3 shows the overlap of one strap attachment 23, one temple pad 3, and one end of the eyewear 1. These components are ideally secured with temporary attachment means such as hook fabric 231 on the strap attachment 23 and loop fabric 31 on the temple pad 3. Snapback fasteners may also be used, for example with male snaps on the strap attachment and female snaps on the temple pad.

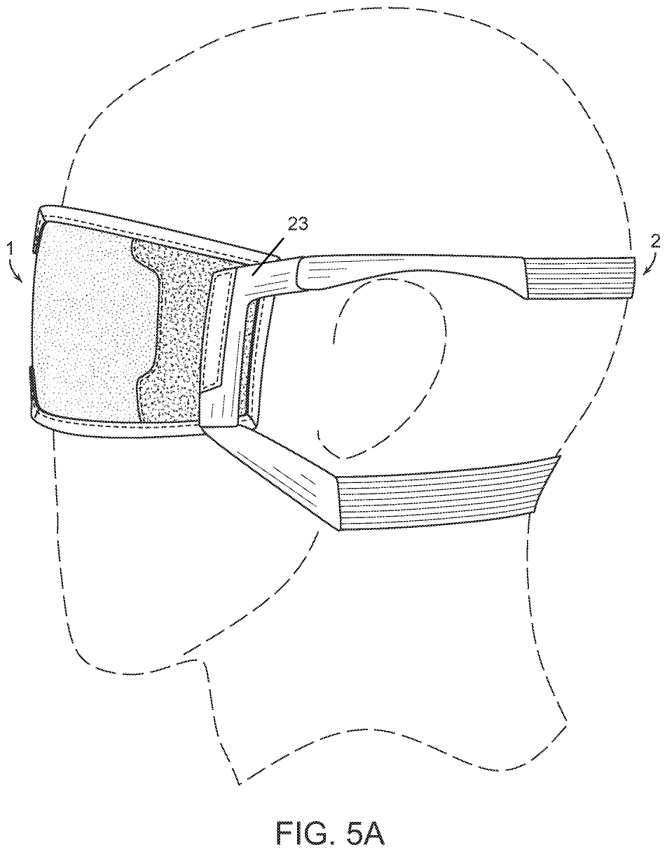

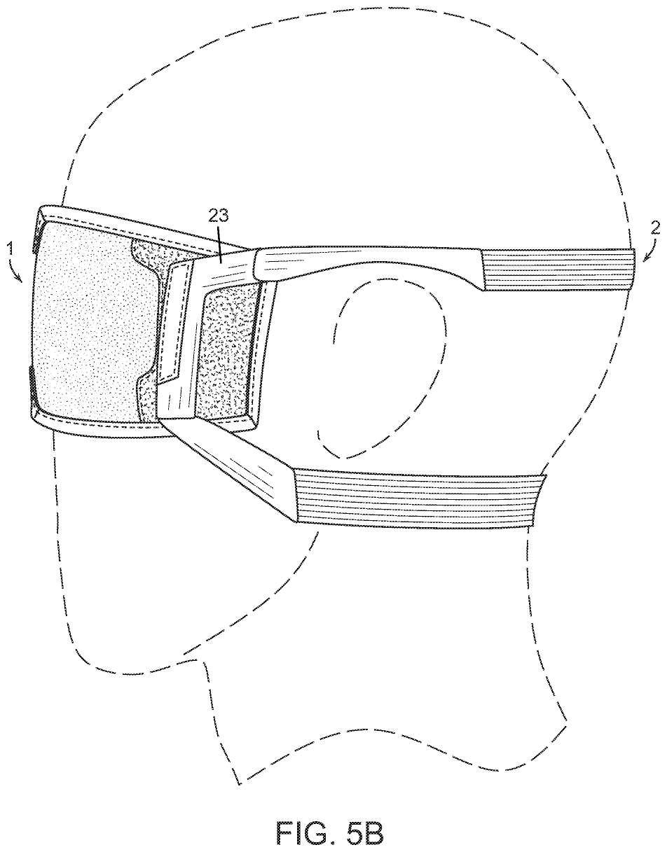

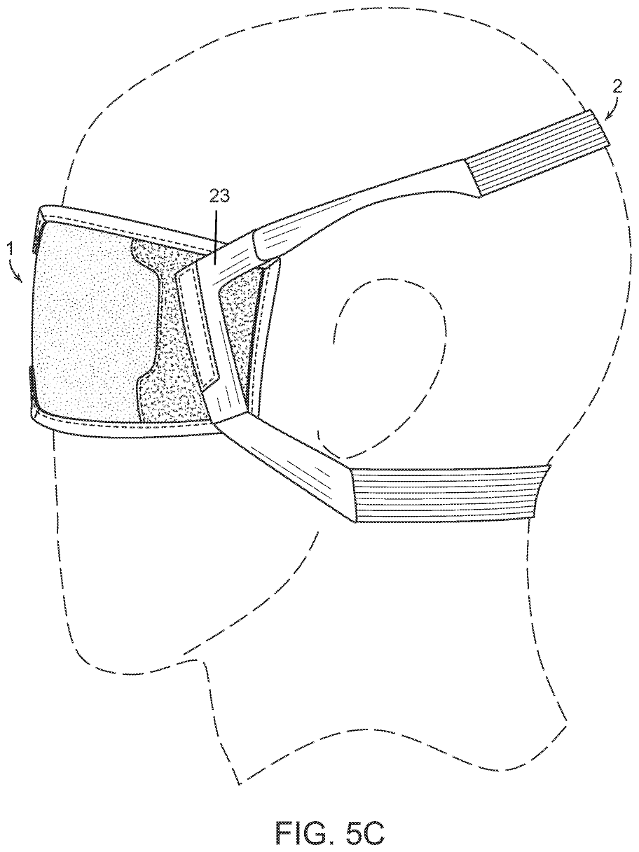

The use of temporary attachment means allows great variation in the placement and orientation of the strap attachments 23, as illustrated in FIGS. 5A, 5B, and 5C. The head strap assembly 2 may be positioned further back on the eyewear 1 for smaller heads (FIG. 5A), or further forward for larger heads (FIG. 5B). The head strap assembly can also be attached to the eyewear at various angles to accommodate ears of different sizes and placements. FIG. 5C shows the head strap assembly 2 angled higher to the rear compared to FIG. 5A or 5B.

An optional chin strap 4 can also be secured to the temple pad 3 with removable attachment means 41 such as hook-and-loop fabric. The chin strap provides extra stability for the eye mask 1. An optional hairline strap 5 (FIG. 4) may also be used.

I claim:

1. A head strap assembly for securing eye wear, comprising:
a central band with a left end and a right end;
a left over-ear strap connected to the left end of the central band, and a right over-ear strap connected to the right end of the central band;
a left strap attachment connected to the left over-ear strap at substantially a right angle, and a right strap attachment connected to the right over-ear strap at substantially a right angle;
a left under-ear strap connected to the left strap attachment, and a right under-ear strap connected to the right strap attachment;
a left neck strap connected to the left under-ear strap, and a right neck strap connected to the right under-ear strap;
and detachable securing means on the left neck strap and the right neck strap, for temporarily securing the left neck strap to the right neck strap.

2. The invention of claim 1, wherein the left over-ear strap and the right over-ear strap are each configured in an arc.

3. The invention of claim 2, further comprising
a left temple pad and a right temple pad;
detachable securing means on the left strap attachment and the left temple pad, for temporarily securing the left strap attachment to the left temple pad;
and detachable securing means on the right strap attachment and the right temple pad, for temporarily securing the right strap attachment to the right temple pad.

4. The invention of claim 3, further comprising eye wear connected to the left temple pad and to the right temple pad.

5. The invention of claim 3, further comprising
a chin strap;
detachable securing means on the chin strap;
detachable securing means on the left temple pad for temporarily securing the chin strap to the left temple pad;
and detachable securing means on the right temple pad for temporarily securing the chin strap to the right temple pad.

6. The invention of claim 5, further comprising
a hairline strap;
detachable securing means on the hairline strap;
detachable securing means on the left temple pad for temporarily securing the hairline strap to the left temple pad;
and detachable securing means on the right temple pad for temporarily securing the hairline strap to the right temple pad.

7. The invention of claim 6, further comprising adjustment means in the central band, for adjusting the central band longer or shorter.

8. The invention of claim 2, wherein
the central band, the left under-ear strap, and the right under-ear strap are each made of elastic material;
and the left neck strap and the right neck strap are each made of neoprene.

9. The invention of claim 6, wherein the detachable securing means on the left strap connector, the right strap connector, the left temple pad, the right temple pad, the chin strap, and the hairline strap are made of hook-and-loop fabric.

10. The invention of claim 9, wherein the detachable securing means on the left neck strap and the right neck strap are made of hook-and-loop fabric.

11. The invention of claim 9, wherein the left neck strap and the right neck strap are snapback straps.

12. The invention of claim 8, in which the left over-ear strap and the right over-ear strap are each made of a plastic insert with a foam cover.

13. The invention of claim 8, in which the left over-ear strap and the right over-ear strap are each made of fabric.

14. The invention of claim 8, in which the left over-ear strap and the right over-ear strap are each made of silicone.
